(19) United States
(12) Patent Application Publication
(10) Pub. No.: US 2007/0015190 A1
(43) Pub. Date: Jan. 18, 2007

(54) Novel bioassay system using a nanoparticle

(76) Inventor: Derhsing Lai, Yardley, PA (US)

Correspondence Address: CHERYL H AGRIS PHD, PO BOX 806, PELHAM, NY, 10803, US

(21) Appl. No.: 11/476,156

(22) Filed: Jun. 28, 2006

Related U.S. Application Data
(60) Provisional application No. 60/695,759, filed on Jun. 30, 2005.

(51) Int. Cl. C12Q 1/68; C12M 1/34; G01N 33/551

(52) U.S. Cl. 435/6; 436/524; 977/924; 435/287.2

(57) ABSTRACT

The invention is directed to a method for detecting a substance of interest using a nanoparticle containing a first substance and a matrix containing a second substance and detecting the radio signal emitted as a result of complex formation between the first and second substance as well as systems and kits using said method.

### FIELD OF THE INVENTION

The invention is directed to a method for detecting a substance of interest comprising detecting a complex of the substance of interest with another substance where one substance is attached to a matrix, surface or substrate containing a means for emitting a radio signal and the other substance is attached to a nanoparticle as well as kits for carrying out the method of the present invention. Either substance may be the substance of interest. The invention is further directed to an assay system comprising a nanoparticle, in particular an integrated circuit nanoparticle read only tag optionally containing a substance and a matrix containing said means for emitting a radio signal.

### BACKGROUND OF THE INVENTION

Solid-Phase Assay Procedures

Solid phase assays have been used to determine the presence and/or amount of substances such as proteins, peptides, carbohydrates, lipids and small molecules in a variety of biological samples (e.g., blood, serum, urine, saliva, tissue homogenates). The solid phase is used to separate molecules that bind to the solid phase from those that do not. Small beads are generally used as the solid phase to capture the analyte. However, in conventional procedures, it is difficult to perform a multiplicity of assays in a single sample at about the same time (multiplex assay).

One approach used involves detecting a particular substance using RFID methods. A radio frequency identification system (RFID) carries information in suitable transponders that contain tags having information. The information on the tags is retrieved in response to a radio signal by machine-readable means (for a review of RFID, see www.aimglobal.org and “Supply Chain Technology”, Bear Stearns Report, June 2003). In one approach, a radiofrequency (RF) encodable microchip is coupled with a polypropylene capsule of derivatized polystyrene resin so that a radioscanner registers the identity of a capsule and the contents of each beaker it enters (see, U.S. Pat. Nos. 5,777,045 and 6,051,377 and Moran, et al., 1995, J. Amer. Chem. Soc. 117:10787-10788). The data is uploaded to a computer that keeps track of the order of addition to monomers to the capsule.

In another approach by Nova et al., the data obtained is actually stored on the microchip itself, using a transmitter that writes the information to the chip (see, for example, U.S. Pat. Nos. 5,741,462, 5,751,629, 5,874,214, 5,925,562 and 6,025,129). The data is not uploaded to the computer until the run is complete. Therefore the system disclosed comprises a recording device and storage unit. It has been suggested that this system may also be used in immunoassays and hybridization reactions and to detect macromolecules, to identify receptor bound ligands, and cell sorting.

US 2004/0029109 discloses IC chips containing read only tags and substances used particularly in bioassays. These tags respond to a radio signal and as a result, emit the logics of photomask embedded material to the receiver. Then, the receiver transfers the logics to the interpreter by means of a data processing machine (computer) to retrieve its data from its data bank in Computer.

Recently, an “organic” polymer based RFID chip has been disclosed (see, for example, (Masselli, “Startup seeks organic RFID chip”, RFID Journal (Sep. 24, 2004), available at www.rfidjournal.com/articleview/851/1/1/1, or www.organicid.com).

However, there are limits to the sensitivity of assays used in these systems.

Nanoparticles

Recently, nanoparticle based systems have been used to detect a variety of substances (Kohli, 2005, Curr. Pharm. Biotechnol. 6:35-47 and Vo-Dinh, 2005, Methods Mol. Biol. 300:1-13). Generally, visual (Crut et al., 2005, Nucl. Acids Res. 20:e98; magnetic (US 2005/0130167, US 2005/0100930) or electrochemical (Wang, 2005, Analyst 130:421-6) detection means have been used.

### SUMMARY OF THE INVENTION

The invention is directed to a method for detecting a substance of interest comprising: (a) providing a first substance attached to a nanoparticle; (b) providing a second substance attached to a substrate, surface or matrix; (c) contacting the first substance with the second substance under conditions suitable for selective binding of the first substance to for a complex and (d) detecting the complex of (c) by means of detecting emission of a radio signal. In a specific embodiment, the nanoparticle contains a read-only tag; in a most specific embodiment, the nanoparticle contains a photomask option. In another specific embodiment, the second substance is attached to a substrate, surface or matrix. The terms “substrate”, “surface” and “matrix” will be used interchangeably and refer to the same material. The matrix will contain a means for detecting emission or transmission of a radio signal, such as an antenna or electrode (e.g., wire or coil).

The substances may be biological substances. In a particular embodiment, the substances are nucleic acid molecules. Alternatively, they may, for example, be protein or peptide molecules, an antibody and antigen or hapten, ligand and isolated receptor or receptor present on a cell or virus, enzyme and substrate, chelator and metal, and polysaccharide and protein.

In the method of the present invention, the formation of a complex between the first and second substance results in the emission of a radio signal that may be detected. Specifically, when a complex is formed, power is transmitted to the IC nanoparticle containing a read only tag through the antenna or electrode on the matrix via radiofrequency. Information on the read only tag is converted to an RF band and is converted to an electromagnetic field.

In a related aspect, the invention is directed to an assay system comprising (a) a nanoparticle containing a read-only tag and/or photomask option that contains unique information (logics) and (b) a matrix, surface or substrate containing a means of emitting a radio signal wherein said read-only tag is activated by said antenna or electrode. The system may further comprise a receiver that receives and decodes information on the read-only tag. Additionally, the matrix and or nanoparticle may contain a substance attached to it.

The invention is further directed to a multiplex assay system comprising a plurality of matrices, present on one surface, where each matrix contains a means for emitting a radiofrequency and where each matrix may optionally contain an attached substance and a nanoparticle containing one or more substances attached to it.

The invention is further directed to kits for use in the method of the present invention. One such kit comprises one or more nanoparticles containing a read only tag and a matrix containing a means for emitting a radio signal. In one embodiment, one or more substances, in particular, biological substances are attached to the nanoparticle(s). In another embodiment, one or more biological substances are attached to the matrix. The kit in another embodiment, may additionally comprise a means for detecting a radio signal, e.g., a receiver.

### BRIEF DESCRIPTION OF THE FIGURES

FIG. 1 shows the hybridization a nano IC tag containing a single stranded nucleic acid molecule (DNA or RNA) with a matrix containing an antenna and single stranded DNA.

FIG. 2 shows the hybridization a nano IC tag containing a single stranded nucleic acid molecule (DNA or RNA) with a matrix containing a wire connection and single stranded DNA.

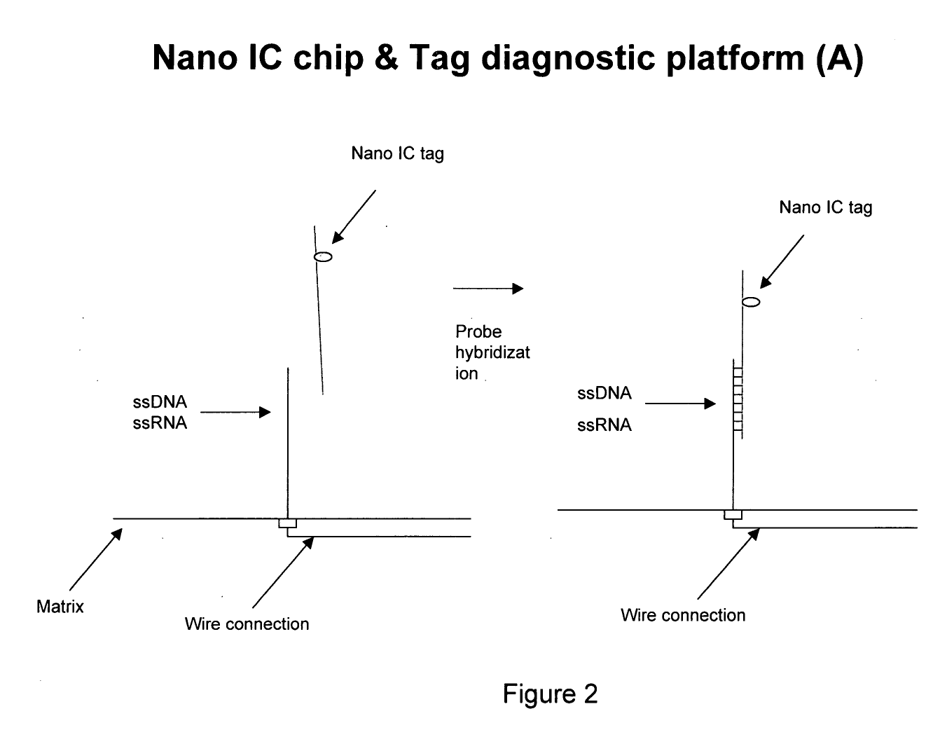

FIG. 3 shows the binding of a nano IC tag containing a ligand with a matrix containing an antenna and a protein, peptide, or chemical.

FIG. 4 shows the binding of a nano IC tag containing a ligand with a matrix containing a wire connection and a protein, peptide, or chemical.

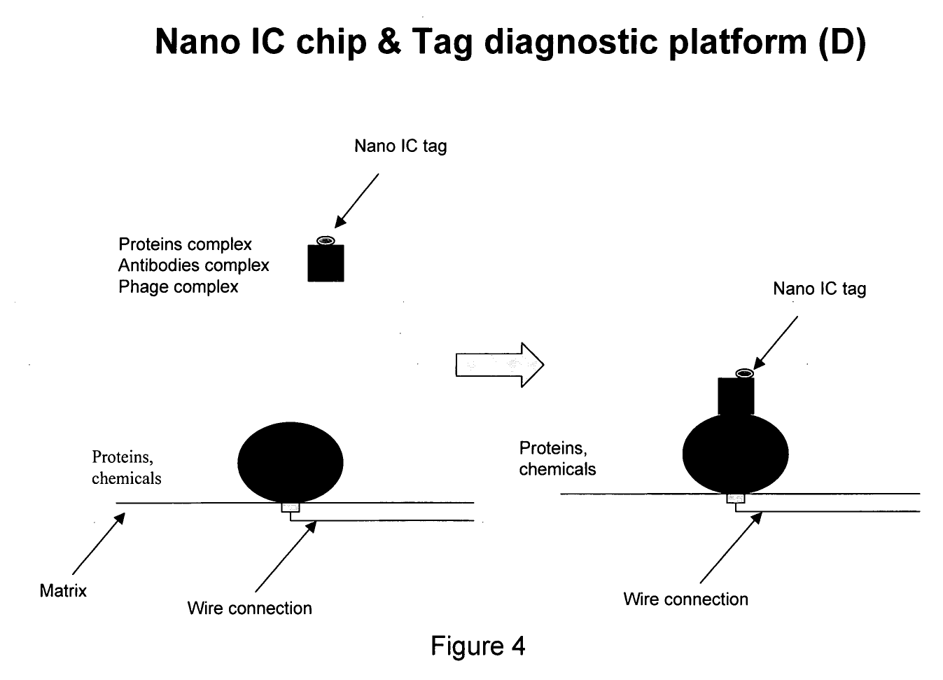

### DETAILED DESCRIPTION OF THE INVENTION

The terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range is encompassed within the invention. The upper and lower limits of these smaller ranges may independently be included in the smaller ranges is also encompassed within the invention, subject to any specifically excluded limit in the stated range. Where the stated range includes one or both of the limits, ranges excluding either both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described.

It must be noted that as used herein and in the appended claims, the singular forms “a,” “and” and “the” include plural references unless the context clearly dictates otherwise.

As defined herein, “nucleic acid molecule” or “polynucleotide” refers to a polymeric form of nucleotides and includes RNA, cDNA, genomic DNA and synthetic forms and mixed polymers of the above. The term, “nucleic acid molecule”, refers to a molecule of at least 10 bases in length. A polynucleotide may contain naturally occurring and/or modified nucleotides linked together by naturally occurring and/or non-naturally occurring nucleotide linkages. Modifications of nucleotides include, but are not limited to, labels, methylation and substitution with an analog.

As used herein, the terms “polypeptide” and “protein” refer to a peptide including more than about 9 amino acid residues connected by peptide linkages.

Matrices

The matrices, substrates or surfaces used in the method of the present invention may be glass, polystyrene, polypropylene, polyethylene, dextran, nylon, natural and modified celluloses, polyacrylamides, and agaroses. In particular, the matrix may also be made of, e.g., plastic created from organic polymers, which contain backbones of carbon atoms linked together or melanin (also see, MCJM Vissenberg, “Opto-Electronic Properties of Disordered Organic Semiconductors”, Ph.D. Thesis, University of Leiden, Jan. 28, 1999, for a review of various organic materials that could be used in the construction of semiconductors).

In one embodiment, the substance, e.g., nucleic acid molecule, protein, polypeptide, antibody, antigen, receptor, ligand is covalently attached to the matrix using a conjugating agent known in the art. Such a conjugating agent includes but is not limited to amino-alkyl silanes [e.g. n-octadecyltrimethoxy-silane (OTMS); n-octadecyltrichlorosilane (OTCS)] [Kleinfeld et al (1988) Neurosci, 8, 4098-4120; Mooney et al, (1996) Proc. Natl. Acad. Sci. USA, 93, 12287-12291], aldehyde silanes, where aldehydes react with primary amines on the proteins to form a Schiff's base linkage [Macbeath et al., (2000) Science 289, 1760-1757]; albumin-alkyl absorption, [Hart et al, (1994) Electroanalysis 6, 617; Newman et al, (1992) Anal. Chim. Acta, 262, 13]; photoresist technology with methyl- and amino-terminated silanes [Britland et al (1992) Biotechnol. Progr, 8, 155-160; Britland et al, (1992) Exp. Cell Res. 198, 124-129]; nitroarylazide photochemistry with biotin-avidin [Pritchard et al, (1995) Anal. Chem., 67, 3605-3607; Hiller et al., (1987) Biochem. J. 248, 167]; perfluorophenylazide photochemistry with n-hydroxysuccinimide esters [Yan et al, (1994) Bioconjugate Chem., 5, 151-157]; diazirine photochemistry [Gao et al, (1995) Bioelectron 10, 317-328]; deep UV of silanes with EDA [Dulcey et al (1991) Science, 252, 551-554]; deep UV of silanes with OTS [Mooney et al., (1996) Proc. Natl. Acad. Sci, 93, 12287-12291]; alkane thios [Knoll et al., (1997) 34, 231-251] and laser vapor deposition [Morales et al., (1995) 10, 847-852].

One agent that can be used for non-specific, non-covalent attachment is poly-L-lysine. The substance to be attached to the matrix will be added to the poly-L-lysine treated or coated chip surface first. The non-specific, non-covalent bond will be formed between the substance and poly-L-lysine. This non-covalent bond will hold the substance on the chip.

In another embodiment, the substance may be coated onto the chip. Specifically, the matrix is directly incubated in a solution containing the substance. The chip is then transferred to the blocking solution (e.g. BSA or casein) [Vogt et al., (1987) J. Immunol. Methods 101, 43-50] to fill the uncovered space on the surface of chip. Non-covalent bonds will be formed between the substance and surface of the chips. The amount of the substance in the coating can be adjusted, depending on the request and the concentration of the substance in the solution.

In yet another embodiment, the matrix may contain a biocompatible coating which may include but is not limited to dextran, dendrimers, amphiphilic polymers/biopolymers (e.g., peptides, phospholipids), silicon oxide, silica, silica-PEG, (His)₆-tag and nickel-nitriloacetic acid. In a particular embodiment, a mixture of several substances can be added to on the single matrix. The positive reaction of the primary screening chip can then be screened for each individual substance from this mixture in each single chip later.

As noted above, the substance attached to the matrix may be a biological substance, which may include but is not limited to a nucleic acid (DNA, RNA, nucleic acid analog), polysaccharide, protein, lipoprotein, lipopolysaccharide, glycoprotein, peptide, cellular metabolite, hormone. In a specific embodiment, members of a DNA or phage display library (e.g, T4 phage) may be attached to a plurality of matrices on a surface (e.g., wells on a plate). The substance may also be an antibody; in a specific embodiment, the antibody is a monoclonal antibody. The substance may also be a receptor or a ligand. A ligand is a substance that binds to a receptor.

The substance may be labeled with a nonradioactive detectable moiety such as a chromophore, fluorophore or luminescent agent. An example of a chromogenic substrate is 5-bromo-4-chloro-3-indoyl phosphate.

Luminescence occurs when a molecule in an electronically excited state relaxes to a lower energy state by the emission of a photon. The luminescent agent in one embodiment may be a chemiluminescent agent. In chemiluminescence, the excited state is generated as a result of a chemical reaction, such as lumisol and isoluminol. In photoluminescence, such as fluorescence and phosphorescence, an electronically excited state is generated by the illumination of a molecule with an external light source. An example of bioluminescence is the enzyme, luciferase. In electrochemiluminescence (ECL), the electronically excited state is generated upon exposure of the molecule (or a precursor molecule) to electrochemical energy in an appropriate surrounding chemical environment. The general principle of ECL is described in Yang et al., 1994, Bio/Technology 12:193-194. Examples of electrochemiluminescent agents are provided, for example, in U.S. Pat. Nos. 5,147,806, 5,641,623 and U.S. application no. 2001/0018187 and include but are not limited to metal cation-liquid complexes, substituted or unsubstituted polyaromatic molecules, mixed systems such as aryl derivatives of isobenzofurans and indoles. The electrochemiluminescent chemical moiety may comprise, in a specific embodiment, a metal-containing organic compound wherein the metal is selected from the group consisting of ruthenium, osmium, rhenium, iridium, rhodium, platinum, palladium, molybdenum, technetium and tungsten.

The matrix of the present invention further comprises a means for emitting a radio signal. This may include an antenna, which may be linear or planar or three dimensional (3D) Alternatively, the matrix may contain an electrode, such as a wire or any metal or material can transmit the electricity. The antenna or wire may be mounted on the matrix using procedures known in the art (see, for example, http://www.alientechnology.com/products/fsa/index.php, http://www.hitachi-eu.com/mu/, http://www.maxell.com/Home/rfid/MaxellRFIDCoil.html.

Nanoparticles

The size of the nanoparticles suitable for use with the present invention is preferably comparable to the size of the target biomolecule to be worked with, such that the nanoparticles do not interfere with biological processes such as DNA hybridization. Consequently, the size of the nanoparticles is preferably from about 5 nm to about 250 nm (mean diameter), more preferably from about 5 nm to about 150 nm, and most preferably from about 5 nm to about 20 nm. For example, nanoparticles having a mean diameter of 5 nm, 6 nm, 7 nm, 8 nm, 9 nm, 10 nm, 11 mm, 12 nm, 13 nm, 14 nm, 15 nm, 16 nm, 17 nm, 18 nm, 19 nm, 20 nm, 25 nm, 30 nm, 35 nm, 40 nm, 45 nm, 50 nm, 55 nm, 60 nm, 70 nm, 80 nm, 90 nm, 100 nm, 110 nm, 120 nm, 130 nm, 140 nm, and 150 nm, as well as nanoparticles having mean diameters in ranges between any two of these values, are suitable for use with the present invention. The nanoparticles may be spherical in shape or alternatively may be in the form of disks, rods, coils, or fibers.

The nanoparticle of the present invention in one embodiment is a silicon semiconductor or integrated circuit (IC) nano chip and is a silicon containing nanoparticle. In another embodiment, it is an organic nanoparticle (see, for example, Masselli, “Startup seeks organic RFID chip”, RFID Journal (Sep. 24, 2004), available at www.rfidjournal.com/articleview/851/1/1/1/) and may be made of, e.g., plastic created from organic polymers, which contain backbones of carbon atoms linked together or melanin (also see, MCJM Vissenberg, “Opto-Electronic Properties of Disordered Organic Semiconductors”, Ph.D. Thesis, University of Leiden, Jan. 28, 1999, for a review of various organic materials that could be used in the construction of semiconductors). The nanoparticle may contain a mask option or layer which may be formed by imprinting, etching or self assembly. In a preferred embodiment, the nanoparticle contains a non-memory containing read only tag. In a most preferred embodiment, the nanoparticle contains a photomask option which is created using procedures known in the art (see, for example, www.photronics.com,). The photomask process results in the creation of an index ID (logics of photomask embedded material) which is converted to a radiofrequency when a complex does form.

As with the matrix, the nanoparticles may be coated with a biocompatible substance. In certain preferred embodiments of the present invention, the biocompatible coating may include but is not limited to dextran, dendrimers, amphiphilic polymers/bio-polymers (e.g., phospholipids and peptides), polymers, silicon oxide, silica, silica-PEG. The biocompatible may be an amphiphilic polymer coating, e.g., a phospholipid-PEG coating. The phospholipid-PEG may be modified with various bioconjugation reactive groups, including, but not limited to amines, maleimide, thiols, carboxylic acids, NHS esters, and the derivatives of these reactive groups to form phospholipid-PEG-X (wherein X is the modified bioconjugation group(s)). In a further preferred embodiment, a mixture of modified phospholipid-PEG molecules with different bioconjugation compatible groups may be used for the coating process.

In certain other preferred embodiments, the coating materials self assemble to form the biocompatible coating. As used herein, a “coating material” refers to a dextran molecule, a dendrimer, an amphiphilic polymer/bio-polymer (e.g. phospholipid, peptide etc.), a polymer or a surfactant. In further preferred embodiments, these self-assembled coating materials form a micelle, liposome, or dendrimer shaped structure. In yet another preferred embodiment, the coating materials form only a monolayer on the surface of a nanoparticle. In a further preferred embodiment, the monolayer thickness can be engineered by controlling the chain length of the self assembled structure (e.g. the PEG chain length may be controlled for a phosoholipid-PEG biocompatible coating). The present invention also provides that the biocompatible coating can be formed by cross-linking or polymerization of raw coating materials to form a network of molecules on the surface of the nanoparticle. In a further preferred embodiment, the crosslinking or polymerization process can be controlled by altering the time of the reactions, the amount of raw coating material, the polymer chain length, the temperature, and other processing conditions.

The attachment of the substance, e.g. nucleic acid probe, protein, to the surface of the nanoparticle may involve a covalent attachment or high affinity adsorption/binding using non-covalent attachment through other biomolecules such as peptides or proteins attached to the nanoparticle coating surface, for example, utilizing a streptavidin-biotin linkage. In one preferred embodiment, the substance, e.g., the nucleic acid molecule is covalently attached to the surface of the nanoparticles through chemical modifications to generate a functional group either at the 3′ end, the 5′ end, or anywhere in the sequence of the probe (i.e. an internal modification of the substance). In a particular embodiment, the substance is attached to the nanoparticle via a (His)₆-tag and/or nickel-triloacetic acid (NTA) system. In a further preferred embodiment, the coated nanoparticle probe is also modified with a luminescent reporter molecule, particularly, an electrochemiluminescent molecule.

Methods and Kits

In one embodiment, the nanoparticle of the present invention is an IC chip containing a read only tag and unique information (e.g., index binary ID; logics of photomask embedded material) created by a photomask process. When the substances present on the nanoparticle and matrix form a complex, the binary ID is converted to a radio signal. The antenna or electrode (e.g., wire or coil) present on the matrix then emits the radio signal to a receiver which receives and decodes the information. Thus, the invention is directed to a system comprising the nanoparticle, matrix and receiver.

In one embodiment, the substance of interest or target substance is present on the matrix and the probe is attached to the nanoparticle. In another embodiment, the substance of interest or target substance is attached to the nanoparticle.

In a particular embodiment, the receiver transmits a signal to the matrix. If a complex is formed between the substance present on the nanoparticle and matrix, the information present on the nanoparticle will be converted to a different frequency from the original frequency transmitted and will be emitted via the antenna or electrode present on the matrix.

In yet another particular embodiment, the matrix may contain both an antenna and electrode, particularly when electrochemiluminescence (ECL) is used. An ECL labeled substance may be attached to the matrix or nanoparticle. An ECL-label such as Tris (2,2′-bipyridine) ruthenium (RU) is coupled to a substance such as DNA, protein or drug and is incubated with a substance that is oxidized, such as tripropylamine (TPA) in a reaction buffer. If a complex forms between substances on the matrix and nanoparticle, an RF voltage will as a aforementioned allowed U.S. patent application (incorporated by substance oxidized (e.g., TPA) are activated by oxidation. The oxidized substance is transferred into a highly reducing agent, which reacts with activated ECL label to create an excited-state form. This form returns to its ground state with emission of a photon at wavelength at 620 nm and long excited state lifetime (˜600 ns) at room temperature. The amount of light produced is directly proportional to the amount of ECL label bound on the IC chip of the present invention and can be captured by the light detection systems (e.g. photo detector, camera, microscope . . . etc.). The production of light indicates the ECL conjugated target binds to the chip powered by RF. Several commercial ECL reader systems are available (e.g. NucleiSens Reader from Organon Teknika company; IGEN).

The invention also provides for multiplex assay systems. In one embodiment, a plurality of nanoparticles and/or a plurality of matrices may be provided to detect two or more complexes simultaneously. In a particular embodiment, a plurality of matrices may be present on one surface, for example, in the form of wells, capillary electrophoresis tube or on a plate. Kits used in the method of the present invention may comprise the nanoparticle containing a read-only tag and matrix containing a means for transmitting or emitting a radio signal. The nanoparticle and/or matrix may contain one or more substances such as a nucleic acid molecule, protein peptide, antigen, hapten, antibody, and small molecule. The kits may further comprise a receiver for detecting radio signals, reagents for carrying out the assays and or detectable labels or reporter moieties (e.g., ECL labels).

### EXAMPLES

Detection of a Target Nucleic Acid

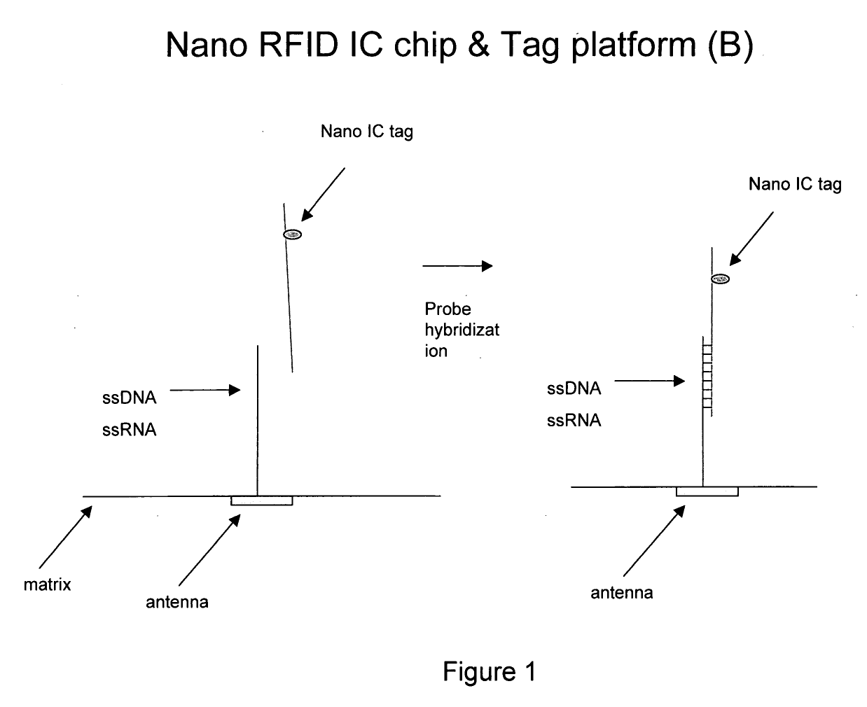

A diagrammatic representation of the procedure used is shown in FIGS. 1 (wire) and 2 (antenna). A nucleic acid probe may be attached to the nanoparticle of the present invention. It may comprise 10-90 nucleotides, more preferably between 10-90 nucleotides, even more preferably between 15-30 nucleotides or 20-25 nucleotides. Alternatively, the probe may comprise larger fragments, e.g., 50, 100, 150, 200, 300, 400, 500, 600, 750, 800, 900, 1000, 1,500, 2000, 3000, 4000, or about 5000 nucleotides. The nanoparticle containing the nucleic acid probe and matrix containing the target nucleic acid sequence are hybridized under stringent conditions known in the art (see, for example, Current Protocols in Molecular Biology, John Wiley & Sons, N.Y. (1989) 6.3.1-6.3.6.). A preferred, non-limiting example of stringent hybridization conditions are hybridization in 6 .times. sodium chloride/sodium citrate (SSC) at about 45 .degree. C., followed by one or more washes in 0.2×SSC, 0.1% SDS at 50-65° C. or alternatively washing with a solution having a salt concentration of about 0.02 molar at pH 7 at about 60° C.

In the event that hybridization occurs, information or code on the nanoparticle is converted to a radio signal emitted from the antenna or wire.

Detection of a Protein

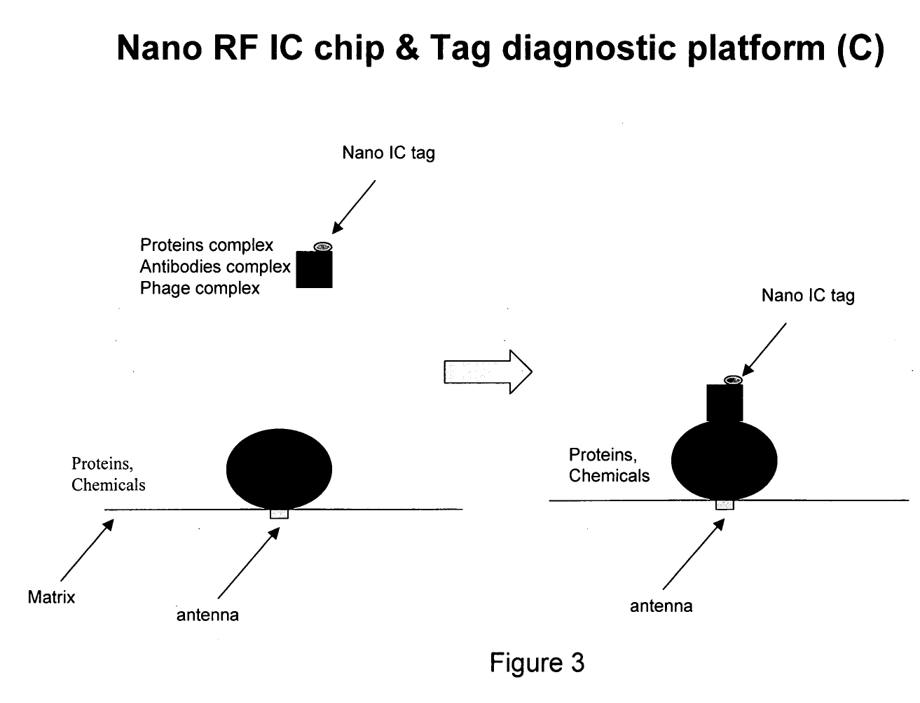

A diagrammatic representation of the procedure used is shown in FIGS. 3 (antenna) and 4 (wire). In a particular embodiment, a nanoparticle containing a read-only tag may be attached to an antibody (e.g., anti-HIV antibody). The antibody or nanoparticle may also contain a detectable label such as an ECL or fluorescent tag. The antibody-nanoparticle is incubated in a reaction vessel with a matrix containing an antenna or wire in the presence of reaction buffer for a period of time sufficient for the antibody to bind to the antigen. The reaction mixture is subsequently washed with washing buffer to remove any uncomplexed antibody. Assays known in the art for detecting a particular antigen or protein using a particular antibody may be used and adapted (see, for example, Ekins, 1987, Clin. Biochem. Revs. 8:12-23 and US 2004/0076948).

Detection of HIV

A sample of human serum is incubated with a well or matrix coated with HIV viral antigens (e.g. gp41, gp24, gp120 . . . etc.) to allow anti-HIV human antibody to bind the HIV viral antigens coated on the well surface. The surface is washed several times with suitable washing buffer to remove unbound material. Anti-human antibody labeled nano-tag is added to the well for 2^{nd} antibody incubation. The surface is washed several times with suitable washing buffer to remove unbound material. The nano-tag can be charged and read with radio-frequency (RF) platform for identification.

The invention described and claimed herein is not to be limited in scope by the specific embodiments herein disclosed, since these embodiments are intended as illustrations of several aspects of the invention. Any equivalent embodiments are intended to be within the scope of this invention. Indeed, various modifications of the invention in addition to those shown and described herein will become apparent to those skilled in the art from the foregoing description. Such modifications are also intended to fall within the scope of the appended claims. Various references are cited herein, the disclosures of which are incorporated by reference in their entireties.

What is claimed is:

1. A method for detecting a substance of interest comprising:
(a) providing a first substance attached to a nanoparticle;
(b) providing a second substance attached to a matrix;
(c) contacting the first substance with the second substance under conditions suitable for selective binding of the first substance to for a complex and
(d) detecting the complex of (c) by means of detecting emission of a radio signal, wherein said substance of interest is present in the complex detected in (d).

2. The method according to claim 1, wherein said second substance is attached to a matrix comprising a means for emitting a radio signal as a result of formation of the complex of step (c).

3. The method according to claim 1, wherein said matrix comprises an antenna.

4. The method according to claim 1, wherein said matrix comprises an electrode.

5. The method according to claim 1 wherein the first substance and/or second substance is a biological molecule.

6. The method according to claim 1, wherein said first substance and/or second substance is a nucleic acid molecule.

7. The method according to claim 1 wherein said first substance and/or second substance is a protein or polypeptide.

8. The method according to claim 1, wherein the first substance is a ligand and the second substance is a receptor.

9. The method according to claim 1, wherein the first substance is a receptor and the first substance is a ligand.

10. The method according to claim 1, wherein the first substance is an antibody and the second substance is an antigen or hapten.

11. The method according to claim 1, wherein said nanoparticle is and integrated circuit (IC) nanoparticle.

12. The method according to claim 1, wherein said nanoparticle is an IC nanoparticle containing a read-only tag.

13. The method according to claim 1, wherein said nanoparticle is an IC nanoparticle containing a photomask option.

14. The method according to claim 1, wherein said first substance or second substance comprises a detectable moiety.

15. The method according to claim 14, wherein said detectable moiety is selected from the group consisting of a chromophore, fluorophore and luminescent agent.

16. The method according to claim 14, wherein said detectable moiety is a luminescent agent selected from the group consisting of a chemiluminescent, photoluminescent, bioluminescent and electrochemiluminescent agent.

17. The method according to claim 4, wherein said second substance is attached to a substrate comprising an electrode and said second substance is contacted with a third substance having an electrochemiluminescent moiety.

18. An assay system comprising
(a) one or more IC nanoparticles containing a read-only tag and/or photomask option which contains unique information;
(b) one or more matrices containing an antenna and/or electrode which emits a radio signal wherein said read-only tag is activated by said antenna or electrode.

19. The assay system according to claim 18, wherein said system further comprises a substance attached to said nanoparticle and/or matrix.

20. The assay system according to claim 18, wherein said system further comprises a receiver which receives the signal from said antenna or electrode.

21. A multiplex assay system comprising
(a) a matrix comprising a plurality of substances;
(b) a means for emitting a radio signal attached to said matrix;
(c) one or more nanoparticles.

22. The system according to claim 21, wherein each nanoparticle is attached to a substance.

23. A method for performing a multiplex assay comprising
(a) providing a plurality of nanoparticles comprising one or more first substances;
(b) providing one or more matrices comprising one or more second substances and a means for emitting a radio signal;
(c) contacting the nanoparticle(s) of step (a) with the substrate of step (b) under conditions suitable for selective binding of at least one first substance to at least one second substance form a complex and
(d) detecting the complex of (c) by means of detecting emission of a radio signal.

24. A kit comprising an IC nanoparticle containing a read-only tag, a matrix containing a means for emitting a radio signal, one or more substances which may be attached to said nanoparticle and/or matrix, means for attaching said substance to said nanoparticle or matrix and optionally a reagent(s) for carrying out the method of claim 1 and a receiver.

25. A kit comprising a substance attached to an IC nanoparticle containing a read-only tag, a matrix containing a means for emitting a radio signal, and optionally a reagent(s) for carrying out the method of claim 1 and optionally a detectable label.
